(19) United States
(12) Patent Application Publication
(10) Pub. No.: US 2014/0294681 A1
(43) Pub. Date: Oct. 2, 2014

(54) NON-THERMAL PLASMA CELL

(71) Applicant: TRI-AIR DEVELOPMENTS LIMITED, London, Greater London (GB)

(72) Inventor: Alan Mole, Worcestershire (GB)

(73) Assignee: TRI-AIR DEVELOPMENTS LIMITED, London, Greater London (GB)

(21) Appl. No.: 14/360,159

(22) PCT Filed: Nov. 14, 2012
(86) PCT No.: PCT/GB2012/052824

§ 371 Date: May 22, 2014

(30) Foreign Application Priority Data
Nov. 25, 2011 (GB) 1120341.1

(51) Int. Cl. A61L 9/22

(52) U.S. Cl. CPC A61L 9/22
USPC 422/121; 29/592.1

(57) ABSTRACT

A non-thermal plasma cell having an annulus of a dielectric material, such as ceramic, formed from a continuous wall of material, the continuous wall having apertures therein, a pair of annular air-permeable electrodes mounted on opposing sides of the wall of the dielectric, and optionally an air gap provided between each electrode and the wall over at a least part of the circumference of the wall. The thickness of the dielectric material is substantially greater than the thickness of the electrodes, which may comprise metal foils.

### PRIORITY CLAIM

This patent application is a U.S. National Phase of International Patent Application No. PCT/GB2012/052824, filed 14 Nov. 2012, which claims priority to United Kingdom Patent Application No. 1120341.1, filed 25 Nov. 2011, the disclosures of which are incorporated herein by reference in their entirety.

### FIELD

The present invention relates to a non-thermal plasma cell, preferably but not exclusively for decontaminating polluted air.

### BACKGROUND

“Non-thermal plasma cells are known. In their simplest form, a non-thermal cell comprises two high-voltage and high-frequency electrodes separated by a space or dielectric which is sufficient to prevent arcing, bui close enough to create an intense electrical field. The dielectric is electrically of a very low conductivity but air within the dielectric is subject to an intense electron bombardment Collisions between the electrons generated by the electric field and the outer ring electrons of the atoms of the component molecules of air create a plasma. This, in the field, is designated ‘nonthermal’ since, although the energy generated by the electron collisions is high, typically around 700 degrees Kelvin and higher, the mass of the electrons is low. Consequently, there is almost no ionisation of the much more massive protons, and the overall temperatures in the plasma remain low, typically in the range of 10 Celsius to 80 Celsius,

When designing a non-thermal plasma cell for use as part of a non-thermal plasma cell filter, an important consideration is the residence time of the air within the cell. If air passes through the plasma too quickly, destruction of any pollutant particles is low, and thus decontamination of the air is poor. However, increasing the residence time of the air within the plasma cell inevitably results in increasing back-pressure, thus requiring far greater energy to maintain an air-flow through the plasma cell. As the back pressure increases, so the energy requirement to force air through the filter and to maintain the plasma logarithmically increases.

Generally, plasma cells are of a flat or rectangular construction, having flat, straight electrode plates with a dielectric sandwiched between the electrodes, see for example, the Applicant's earlier Patent GB2415774B. Other tubular plasma cells have also been considered, such as that described in the Applicant's Published PCT Application No. 2008/074969. However, these types of plasma cell have been found to be problematic in air decontamination devices for a number of reasons, most notably the high cost of manufacture of the plasma cell, the variable efficiency of the cell and the noise created as a result of the frequency of the applied power being in the audible range for humans and animals.

It is an object of the present invention to provide a non-thermal plasma cell, particularly but not exclusively for decontaminating polluted air, that aims to overcome, or at least alleviate, the abovementioned drawbacks.

Accordingly, a first aspect of the present invention provides a non-thermal plasma cell comprising:
an annulus of a dielectric material formed from a continuous wall of material, said continuous wall having a plurality of apertures therein; and
a pair of annular air-permeable electrodes mounted on opposing sides of the wall of the dielectric.

Preferably, the thickness of the dielectric material is substantially greater than the electrodes. The actual thickness of the electrodes is dependent upon the resistance of the material and the current applied but preferably the thickness of each electrode is a maximum of a tenth of the thickness of the dielectric material. In practice, a ratio of dielectric: electrode of 30:1 has been found to be effective using a stainless steel electrode. The diameter of the apertures may be varied to suit a particular application and be optimised having regard to the dielectric dimensions. Generally, the apertures will be 2.5-3.5 mm in diameter. For example, a 3 mm dielectric preferably uses a 3 mm diameter aperture. The diameter can be varied to suit different air flows and applied energy and to alter the characteristics of the plasma cell.

The apertures preferably extend perpendicularly to the circumference of the annulus. The apertures preferably comprise cylindrical holes but alternative designs may be used, such as longitudinal slots extending partially or fully around the circumference of the dielectric. Preferably multiple rows of the plurality of apertures are provided on each annulus. In a preferred embodiment, adjacent rows are staggered, preferably by 2-10 degrees, more preferably by 6 degrees. This aids the manufacture of the device using injection moulding techniques.

It is to be appreciated that the number and dimensions of the apertures will depend upon the size and power of a particular plasma cell.

Preferably, an air gap is provided between each electrode and the wall over at a least part of the circumference of the wall.

The air gap between the dielectric and the electrode is preferably 0.1-2 mm, more preferably 0.2-1 mm, especially 0.4-0.6 mm.

The dielectric may be any appropriate material having the required physical and electrical properties. Preferably, the dielectric is ceramic, such as fired ceramic or partially fired ceramic. Alternatively, the dielectric may comprise a pressed mineral, for example comprising alumina and titanium dioxide, glass fibres or a coarse glass wool. The dielectric may also be formed from a paper or card material impregnated with alumina and titanium dioxide.

In a preferred embodiment of the present invention, the dielectric is formed by injection moulding.

One or more longitudinal grooves or recesses may be provided around all, or part of, the circumference of the dielectric. The grooves serve to increase the retention time of the air stream within the cell thereby increasing turbulence in the air flow. However, there is a consequential negative effect on back pressure. This may be deemed acceptable for a particular design for a specific application where a contaminant molecule is to be removed.

The electrodes are formed from any conductive material that is permeable to air, such as metal mesh or a metal profile sheet. Preferably, each electrode comprises a thin sheet of material having a plurality of apertures therein. Each electrode preferably comprises foil sheets of material, preferably being fainted by means of acid etching.

Preferably, each electrode comprises stainless steel. Alternatively, wire mesh may be used.

In one embodiment of the present invention, the plasma cell is open-ended, i.e. in the form of a ring. Alternatively, one end of the annulus may be closed, i.e. in the form of a cylinder having a base extending between one edge of the continuous wall. The base serves a mechanical function.

The electrodes fit tightly to the inner and outer wall of the dielectric, preferably forming an interference fit between the electrodes and dielectric such that no additional fixings are required. Electrical contacts preferably form an integral part of one or both electrodes.

It is to be appreciated that multiple plasma cells according to the present invention may be combined to provide a plasma field of any required dimension. The modular nature of such a plasma cell also enables the multiple-cell unit to continue working should one of the cells fail.

According to a second aspect of the present invention, there is provided a method of manufacturing a non-thermal plasma cell comprising forming an annulus of a dielectric material comprising a continuous wall having a plurality of apertures therein and attaching an annular air-permeable electrode to opposing sides of said wall.

It is preferable for an air gap to be established between the electrode and the dielectric.

Preferably, the dielectric is injection moulded into the required shape with the desired number and pattern of apertures and the electrodes are then attached to each side of the wall.

Preferably, each electrode is in the form of a sheet material, preferably having a plurality of apertures therethrough. More preferably, the pattern of apertures is formed by acid etching a sheet of conductive material.

In an alternative embodiment of the second aspect of the present invention, the dielectric is formed from a sheet of paper or cardboard impregnated with a dielectric such as alumina with titanium dioxide. An appropriate pattern of apertures is provided through the sheet and the electrode sheets are printed on to the dielectric prior to forming the annular ring.

The non-thermal plasma cell according to the first aspect of the present invention is particularly suitable for use in relation to air decontamination wherein polluted air is passed through an activated plasma cell so that free radicals are produced by which contaminants in the airstream are neutralised.

To this end, a third aspect of the present invention provides an air decontamination device comprising a housing having an air inlet, an air outlet and an air flow passage therebetween, the housing including a non-thermal plasma cell according to the first aspect of the present invention positioned in the air flow passage.

Preferably, the air decontamination device includes one or each of a UV radiation emitting device, an ozone catalyzing device and a hydrocarbon emitter.

It is preferred that the UV radiation emitting device is positioned within a central region of the annulus of the non-thermal plasma cell whereby the plasma field generated in this region by the plasma cell causes emission of the radiation without need for a separate power source for the UV radiation emitting device.

For a better understanding of the present invention and to show more clearly how it may be carried into effect reference will now be made, by way of example only, to the accompanying drawings in which:

FIG. 1 is perspective view of a dielectric for a non-thermal plasma cell according to one embodiment of the present invention;

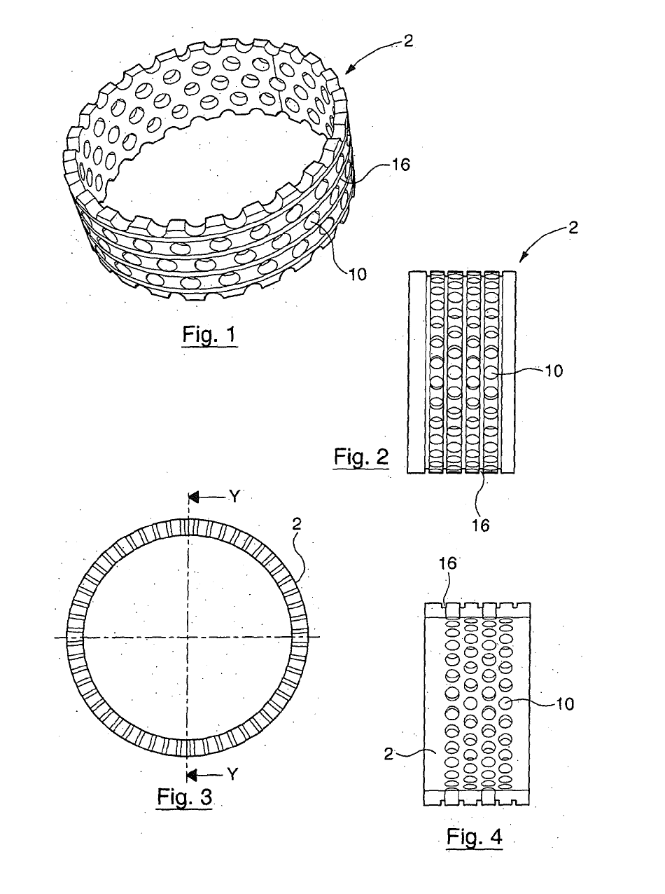

FIG. 2 is an external side view of a dielectric for a non-thermal plasma cell according to another embodiment of the present invention;

FIG. 3 is a top plan view of the dielectric shown in FIG. 2;

FIG. 4 is a section along Y-Y of the dielectric shown in FIG. 2;

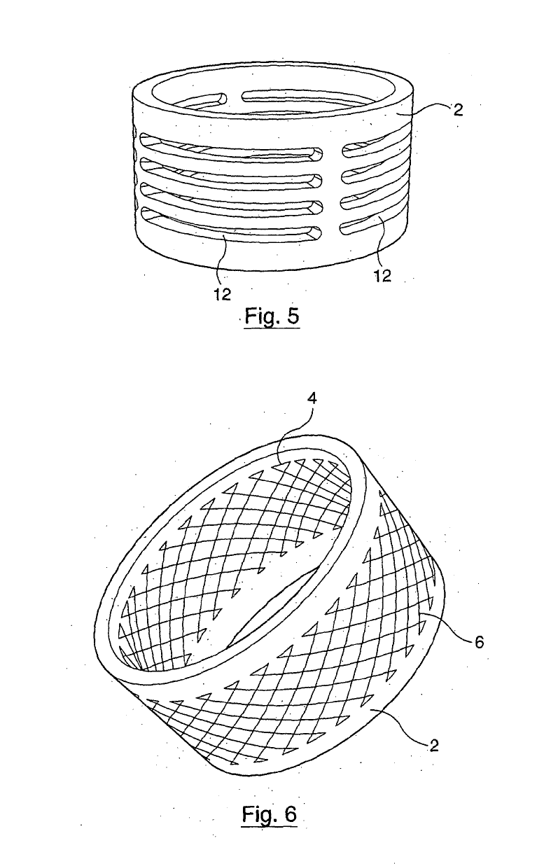

FIG. 5 is a perspective view of a dielectric for a non-thermal plasma cell according to a further embodiment of the present invention;

FIG. 6 is a perspective view of a non-thermal plasma cell comprising a dielectric and electrodes according to an embodiment of the present invention;

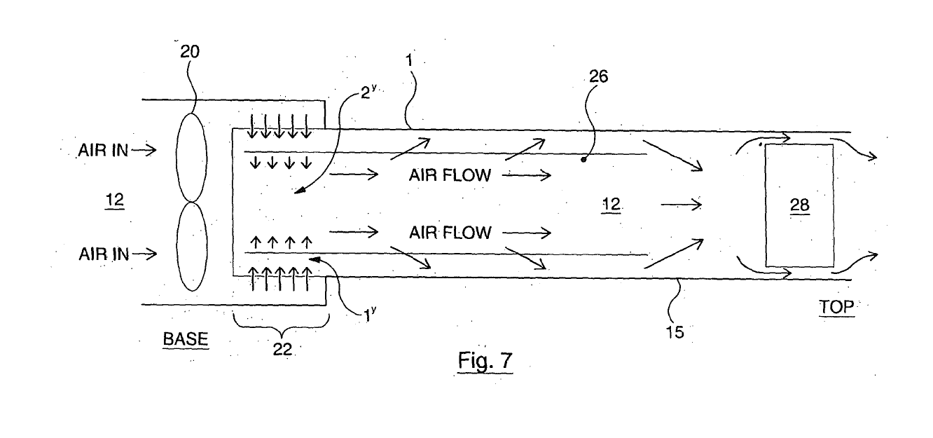

FIG. 7 is a schematic diagram of an air decontamination device incorporating a plasma cell according to an embodiment of the present invention;

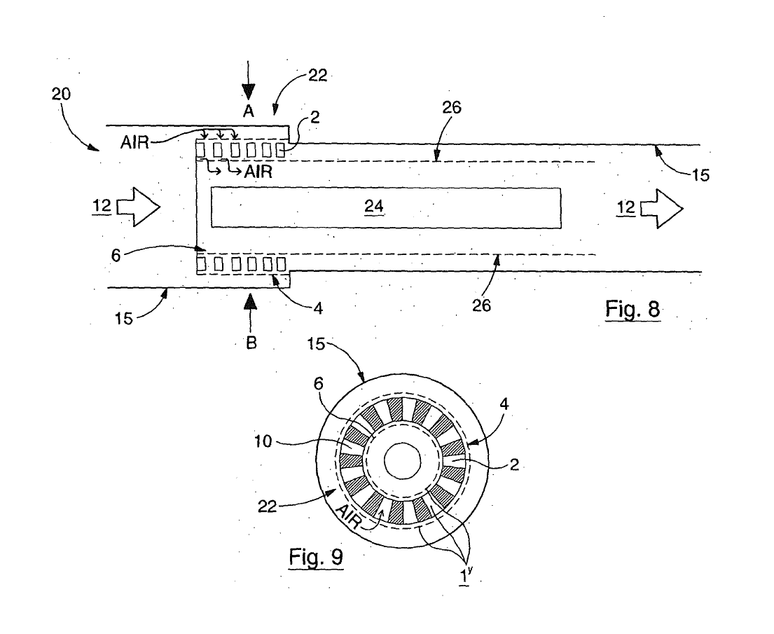

FIG. 8 is a longitudinal cross-sectional view of an air decontamination device incorporation a plasma cell according to an embodiment of the present invention;

FIG. 9 is a traverse cross-sectional view along line A-B of the device shown in FIG. 8; and

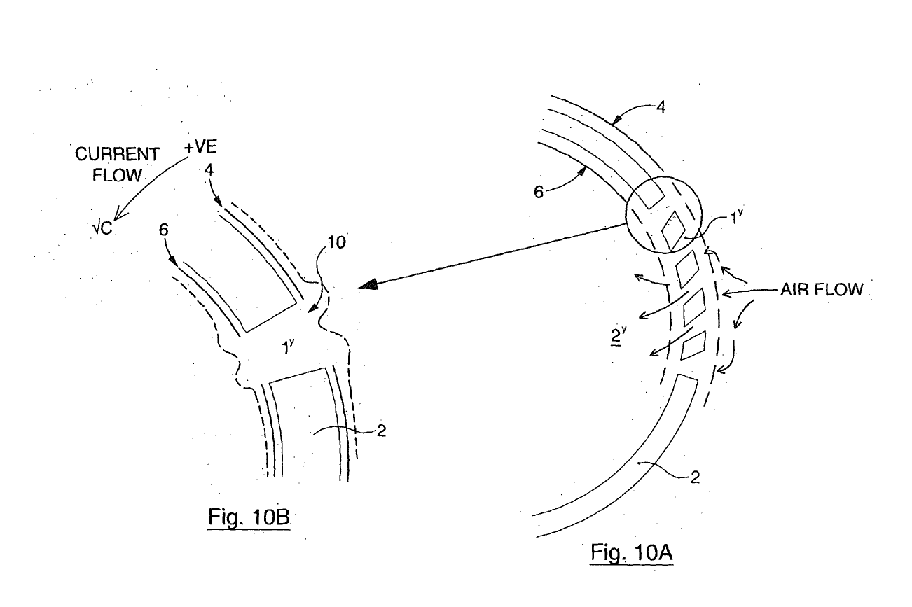

FIGS. 10A and 10B illustrate air flow and plasma generation in a non-thermal plasma cell according to the present invention.

FIGS. 1, 2 to 4 and 5 of the accompanying drawings illustrate different types of dielectrics for incorporation into a non-thermal plasma cell according to the present invention. The dielectric comprises an injection moulded ceramic ring 2 having a plurality of circular holes 10 therethrough. Grooves 16 may be provided at intervals around the circumference of the dielectric, as shown in FIGS. 2 to 4.

The inner and outer faces of the ceramic ring have a stainless steel annular foil 4, 6 mounted thereon, the foils being etched of material by acid etching to faun a plurality of holes through the foil, as illustrated in FIG. 6. The ring may be any suitable diameter with the dielectric preferably being approx. 3 mm thick. The apertures have a diameter of approx. 3 mm and the foil is a fraction of the thickness of the ceramic ring (preferably being at least a tenth of the thickness of the ring).

The circumference of the foils is such that they form an interference fit around the central ceramic ring thereby removing the need for separate fixing means but are spaced slightly away from the surface of the ring to form a small air gap of around 0.2-1 mm, preferably 0.5 mm. The foils terminate in a contact for attachment to a power source (not shown).

The non-thermal plasma cell is produced from relatively cheap components and may be manufactured easily in bulk quantities. The arrangement of the dielectric with respect to the electrodes also increases residence time of air within the non-thermal plasma cell without significantly increasing back-pressure, thereby increasing the efficiency of the plasma cell.

FIG. 5 of the accompanying drawings illustrates an alternative embodiment of a dielectric for a plasma cell according to the present invention. The plasma cell again comprises an annual ceramic dielectric ring 2 but instead of circular holes the plasma cell has a series of longitudinal slots 12 around its circumference. Again, sheets of metal electrodes (not shown) are wrapped around the inner and outer wall of the dielectric. This embodiment allows for increased airflow for a higher power input.

The annular plasma cell according to the present invention allows for infinite variations in dimensions, resulting in an infinite amount of plasma power being possible. It is to be appreciated that an annular cell having a single row or multiple rows of perforations may be combined with any number of similar cells to provide the required dimensions. A wide range of input frequencies may also be used with the device of the present invention, from 1 kHz to 50 kHz or more, whereas conventional plasma cells only operate within a relatively narrow frequency range of 1-10 kHz. Component power supplies are readily available in this range, originally being intended for commercial ozone generation. This also enables a frequency to be chosen which is not within the audible range of humans and animals.

Rapid switching of frequencies may be possible. This may be achieved using any suitable proprietary system such as a Blumlein switch. A Blumlein switch is composed of a loop of co-axial cable and a discharge gap, different size loops and gaps providing different switched frequencies. The rate at which such switching may take place may be as high as several hundred thousand times per second.

The plasma cell is simple and cheap to manufacture in large quantities, using less raw materials and more recyclables compared with the prior art. Manufacture may involve injection moulding the annular dielectric ring followed by attachment of the electrodes that have been produced by acid etching. The plasma cell according to the invention provides very little back pressure enabling more efficient operation of a machine incorporating the device.

Alternatively, the dielectric may be manufactured from reinforced paper or card impregnated with alumina and titanium dioxide. A sheet of the impregnated paper or card is then printed with the metal electrode on each facing surface and formed into the ring.

FIGS. 7 to 9 of the accompanying drawings illustrate a non-thermal plasma cell according to the present invention incorporated into an air decontamination device. This is the preferred application for the non-thermal plasma cell according to the present invention but its use is not limited to this application. The air decontamination device comprises a housing 15 having a flow passage 12, an air inlet to the flow passage 12 and an air outlet exiting from the passage 12. The housing includes an air stream generator 20 (such as a fan), a non-thermal plasma filter 22 according to the invention, an ultraviolet (UV) radiation emitting device 24 (omitted in FIG. 7), an ozone catalysing device 26, and a hydrocarbon emitter 28 (shown only in FIG. 7) located in the passage 12.

The air stream generator 20 is provided adjacent the air inlet of the passage 12. The air stream generator 20, in this embodiment, is an electric fan powered by mains electricity or battery packs (not shown) provided in the compartment of the housing 15. As a safety measure, a grill may be provided across the air inlet to prevent accidental access to the fan 20 while in operation.

The non-thermal plasma filter 22 is positioned adjacent the fan 20, downstream of the air inlet. The plasma filter 22 comprises an annular ring of dielectric 2 having sheet electrodes 4, 6 attached to each side, as described in FIGS. 1 to 6. The plasma cell is orientated within the housing such that air flows through the wall of the annular ring and then passes up through the centre of the ring. The electrodes are powered by a power supply unit (not shown) housed in a compartment of the housing 15. Optionally, the dielectric material may be coated with a catalytic material.

The plasma cell may be configured from any number of rings of dielectric with surrounding electrodes to provide a wide range of frequency output thereby enabling the ozone output from the cell to be controlled.

The UV radiation emitting device 24 includes an ultraviolet light emitting tube which is disposed within the central region of the ring of the non-thermal plasma cell 22, and the ozone catalysing device 26 surrounds the UV-emitting tube. The ozone catalysing device 26 comprises a mesh that includes a coating of ozone catalysing material, such as a mixture of titanium, lead and manganese oxides.

The hydrocarbon emitter 28 includes a rechargeable hydrocarbon reservoir located in a compartment of the housing 15, an evaporator for evaporating liquid hydrocarbon held in the reservoir, and a pump by which the gaseous hydrocarbon is discharged into the passage 12. The various parts of the emitter 28 are omitted from the drawings for the sake of clarity. The reservoir contains a liquid aromatic hydrocarbon, for example an olefin such as a Terpene and, more specifically, Myrcene. The outlet of the hydrocarbon emitter 28 is located at or in the vicinity of the centre of the passage 12 of the housing 15, and downstream of the UV light emitting tube 24 and mesh 26 of the ozone catalysing device. The outlet of the hydrocarbon emitter 28 is located adjacent the outlet of the passage 12 of the housing 15.

Any other suitable means for supplying volatised aromatic hydrocarbon to the outlet of the hydrocarbon emitter 28 can be used.

The air decontamination device can be solely powered by mains electricity, solely powered by battery packs, which may be rechargeable, or may be selectively energisable by both power sources.

The air decontamination device can be produced in the form of a portable device, and this can take the dimensions of or substantially of a suitcase. Alternatively, the air decontamination device can be produced as a larger device intended to remain in one location once installed. The latter device is more suitable for, but not limited to, industrial or commercial installations and premises.

In use, the air decontamination device is positioned in the location to be decontaminated. The device is intended to decontaminate air within a building, chamber, enclosure, trunking, pipe, channel or other enclosed or substantially enclosed area. However, with sufficient through-flow capacity, it can also decontaminate air in an open outside environment. In this respect, air that has passed through the device is able to continue decontamination of the surrounding air once it has exited from the housing.

The device is energised, and the fan 20 generates a stream of ambient air along the passage 12 of the housing 15. The air stream passes initially through the non-thermal plasma filter 22. The filter utilises the characteristics of a non-thermal plasma to ‘plasmalise’ the constituent parts of the air within the dielectric core. In general terms, the outer ring electrons in the atomic structure of the elements comprising air (principally oxygen and nitrogen) are ‘excited’ by the intense electronic field generated by the non-thermal plasma, typically being up to 40 kV and 45 kHz.

FIGS. 10A and 10B of the accompanying drawings illustrate the air flow and plasma generation within the device 15. Pressure from the fan 20 creates high pressure outside of the plasma cell 22 and air flows across the surface of the electrode and then through the voids in the dielectric and inner electrode. Air flows in the same direction as the current flow unlike the devices of the prior art where air flow is against the current. A primary (1^{y}) plasma is created in the immediate vicinity of the electrodes and dielectric (illustrated by the dashed lines in FIG. 10B) and a secondary (2^{y}) plasma is induced in the centre of the annular ring. The greater the current supplied, the greater the intensity of the primary and secondary plasmas.

The energised electrons in the plasma regions release energy through collisions. However, little or no heat is emitted due to the insubstantial mass of the electrons and the consequent lack of ionisation that occurs. The released energy is sufficient to generated free radicals within the air stream, such as O¹⁰⁸ and OH⁻. The free radicals are powerful oxidants, and will oxidise hydrocarbons, organic gases, and particles typically 2.5 picometres and below, such as bacteria, viruses, spores, yeast moulds and odours. Only the most inert elements or compounds will generally resist oxidation.

Since many of the resultants of the oxidative reactions are transient and surface acting, due to having zero vapour pressure, by providing a molecular thick catalytic coating on some or all of the dielectric material of the non-thermal plasma, oxidation of particular molecules or compounds, for example nerve gas agents, within the non-thermal plasma can be targeted.

The non-thermal plasma filter 22 produces ozone as one of the by-products. This is entrained in the air stream leaving the non-thermal plasma filter 22. The half-life of ozone is dependent on atmospheric conditions and, itself being a powerful oxidant, under normal circumstances will continue to react in the air long after it has exited the plasma core. This is unacceptable for a device operated by and in the general vicinity of people.

The air stream passing through the non-thermal plasma filter 22 therefore passes to the UV light emitting tube 24 and through the mesh 26 of the ozone catalysing device. The ultraviolet radiation emitted at 253.4 nanometres wavelength by the 1.1V light emitting tube acts to break down the ozone entrained in the air stream leaving the plasma filter 22. The coating on the mesh 26 acts to catalyse this break down. The provision of a secondary plasma field 2^{y} created in the inner part or central region of the annular ring may be used to excite mercury provided in a mercury vapour tube to emit the UV radiation, in addition to decontamination of the air. This means that a separate power source for the UV emitter is not required, drastically reducing the cost of the air decontamination device.

This destruction (photo-oxidation) of the ozone increases the free radical level, and particularly the level of Hydroxyl radicals OH⁻, within the air stream. These free radicals also vigorously oxidise contaminants remaining within the air stream.

Trials have shown that free radicals resident in the air stream post-plasma filtering significantly increase the rate of generation of free radicals during the photo-oxidative process. Thus, the device provides a cascade effect whereby air exiting from the outlet continues to decontaminate air outside of the device.

1. A non-thermal plasma cell, comprising:
an annulus of a dielectric material formed from a continuous wall of material, said continuous wall having a plurality of apertures therein; and
a pair of annular air-permeable electrodes mounted on opposing sides of the wall of the dielectric.

2. The non-thermal plasma cell of claim 1, wherein an air gap is provided between each electrode and the wall over at a least part of the circumference of the wall.

3. The non-thermal plasma cell of claim 1, wherein the thickness of the dielectric material is substantially greater than the thickness of the electrodes.

4. The non-thermal plasma cell of claim 1, wherein multiple rows of the plurality of apertures are provided on each annulus and the rows are staggered with respect to an adjacent row.

5. The non-thermal plasma cell of claim 1, wherein the dielectric is a material selected from the group consisting of a ceramic, a pressed mineral comprising alumina and titanium dioxide, and a coarse glass wool.

6. The non-thermal plasma cell of claim 1, wherein the dielectric is formed from a paper or card material impregnated with alumina and titanium dioxide.

7. The non-thermal plasma cell of claim 1, wherein each electrode comprises a thin sheet of material having a plurality of apertures therein.

8. The non-thermal plasma cell of claim 7, wherein each electrode comprises a foil sheet of material formed by means of acid etching.

9. The non-thermal plasma cell of claim 1, wherein each electrode forms an interference fit with the inner and outer walls of the dielectric material respectively.

10. The non-thermal plasma cell of claim 1, wherein electrical contacts form an integral part of one or both electrodes.

11. The non-thermal plasma cell of claim 2, wherein the air gap between each electrode and the dielectric is 0.1-2.0 mm.

12. A method of manufacturing a non-thermal plasma cell, the method comprising:
forming an annular ring of a dielectric material comprising a continuous wall having a plurality of apertures therein; and
attaching an annular air-permeable electrode to opposing sides of said wall.

13. The method of claim 12, further comprising including an air gap between the electrode and the dielectric.

14. The method of claim 12, further comprising injection moulding the dielectric into the required shape with the desired number and pattern of apertures and then attaching the electrodes to each side of the wall.

15. The method of claim 12, further comprising forming the dielectric by impregnating a sheet of paper of cardboard with a dielectric material, providing an appropriate pattern of apertures through the sheet and printing the electrodes onto opposing surfaces of the wall of the dielectric prior to forming the annular ring.

16. An air decontamination device, comprising a housing having an air inlet, an air outlet and an air flow passage therebetween, the housing including the non-thermal plasma cell of claim 1 positioned in the air flow passage.

17. The air decontamination device of claim 16, further comprising at least one of a UV radiation emitting device, an ozone catalyzing device and a hydrocarbon emitter.

18. The air decontamination device of claim 17, wherein the UV radiation emitting device is positioned within the annulus of the non-thermal plasma cell whereby the plasma field generated in this region by the plasma cell causes emission of the radiation without need for a separate power source for the UV radiation emitting device.
